(19) United States
(12) Patent Application Publication
(10) Pub. No.: US 2025/0051268 A1
(43) Pub. Date: Feb. 13, 2025

(54) METHOD FOR PREPARING ISOCYANATE BY MEANS OF GAS-PHASE SOLVENT-FREE METHOD

(71) Applicant: MOJIA (SHANGHAI) BIOTECH CO., LTD., Shanghai (CN)

(72) Inventors: Yonghe XUE, Shanghai (CN); Man Kit LAU; Chengliang LU, Shanghai (CN); Haixin YUAN, Shanghai (CN); Ansen CHIEW

(21) Appl. No.: 18/720,677

(22) PCT Filed: Dec. 17, 2021
(86) PCT No.: PCT/CN2021/139210

§ 371 Date: Jun. 17, 2024

(51) Int. Cl. C07C 263/10

(52) U.S. Cl. CPC C07C 263/10

(57) ABSTRACT

The present application relates to a method for preparing an isocyanate. Specifically, in the method of the present application, phosgene (especially liquid phosgene) is used as a quench medium to prepare isocyanate.

### FIELD OF THE INVENTION

The present invention relates to a method for preparing an isocyanate, and more specifically, to a method for preparing an isocyanate by using phosgene (particularly, liquid phosgene) as a quenching medium.

### BACKGROUND ART

Isocyanates are a class of compounds containing one or more isocyanate groups, including aliphatic isocyanates, aromatic isocyanates, unsaturated isocyanates, halogenated isocyanates, thioisocyanates, phosphorus-containing isocyanates, inorganic isocyanates and blocked isocyanates. Because of the highly unsaturated isocyanate groups contained, they are highly chemically active and can undergo important chemical reactions with many substances. Therefore, isocyanates are widely used in polyurethane, polyurethane-urea, polyurea, polymer modification, reagents for organic synthesis, agriculture, medicines and other fields.

In the prior art, the principle underlying the preparation of an isocyanate with phosgene and an amine is well known, and the method mainly includes a liquid phase method and gas phase method. In the process of preparing an isocyanate by a gas-phase method, the isocyanate formed in the reactor is thermally unstable at a high reaction temperature (for example, 300˜400° C.). Therefore, the obtained reaction product mixture is required to be quickly cooled to a temperature below 200° C. after the phosgenation reaction, to avoid the formation of undesirable by-products due to the thermal decomposition of isocyanate or further reactions. To this end, an organic solvent (e.g., toluene, chlorobenzene and chloronaphthalene) (see CN102245565B), an isocyanate (see CN110914236A) or a mixture of a solvent and an isocyanate (see CN110072845A) is often used as a quenching medium to condense the reaction product mixture in the prior art. One of the disadvantages of using these quenching media is that a solid deposit is likely to be formed in the reactor, which eventually blocks the passage of the gaseous reaction product mixture. Accordingly, the reactor is required to be shut down to clean the reaction passage. Moreover, the recovery of the organic solvent also increases the production cost. In the prior art, to reduce the formation of the solid deposit as much as possible, a quenching liquid is injected into a quenching zone by passing the quenching liquid through a quenching liquid nozzle arranged at an inlet of the quenching zone (see CN111094240A). This raises a higher equipment requirement for the reactor and increases the production cost.

Therefore, there is still a need for an optimized method for preparing an isocyanate.

### SUMMARY OF THE INVENTION

An object of the present application is to provide a method for preparing an isocyanate, and more specifically, to a method for preparing an isocyanate by using phosgene (particularly, liquid phosgene) as a quenching medium or by adjusting the ratio of flow rates of the quenching medium to a phosgene stream before the phosgenation reaction.

In one aspect, the present application provides a method for preparing an isocyanate, which includes the following steps:
(a) providing a reactant amine stream and a phosgene stream, f being passed through and reacting in a reaction zone of a first vessel at a temperature of 200° C.˜600° C., to obtain a reaction product mixture comprising isocyanate, hydrogen chloride and unreacted phosgene;
(b) contacting the reaction product mixture obtained in Step (a) with a first quenching medium stream introduced into a quenching zone of the first vessel, and introducing, via an inlet of a second vessel connected to the quenching zone of the first vessel, the reaction product mixture into the second vessel;
(c) the second vessel of Step (b) comprises a collecting zone and a washing zone, collecting isocyanate in the collecting zone, and passing the hydrogen chloride, unreacted phosgene, and uncollected isocyanate through the washing zone; and
(d) introducing a second quenching medium stream into the washing zone of the second vessel of Step (c), and contacting the hydrogen chloride, unreacted phosgene and uncollected isocyanate of Step (c) with the second quenching medium stream in the washing zone.

The first quenching medium and the second quenching medium are independently selected from the group consisting of isocyanate, phosgene, hydrogen chloride, an inert carrier gas, and any combination thereof, and the ratio of flow rates of the first quenching medium to the phosgene stream of Step (a) is from 0.4:1 to 2:1.

In another aspect, the present application provides a method for preparing isocyanate, which includes the following steps:
(a) providing a reactant amine stream and a phosgene stream, being passed through and reacting in a reaction zone of a first vessel at a temperature of 200° C.˜600° C., to obtain a reaction product mixture comprising isocyanate, hydrogen chloride and unreacted phosgene;
(b) contacting the reaction product mixture obtained in Step (a) with a first quenching medium stream introduced into a quenching zone of the first vessel, and introducing, via an inlet of a second vessel connected to the quenching zone of the first vessel, the reaction product mixture into the second vessel;
(c) the second vessel of Step (b) comprises a collecting zone and a washing zone, collecting isocyanate in the collecting zone, and passing the hydrogen chloride, unreacted phosgene, and uncollected isocyanate through the washing zone; and
(d) introducing a second quenching medium stream into the washing zone of the second vessel of Step (c), and contacting the hydrogen chloride, unreacted phosgene and uncollected isocyanate of Step (c) with the second quenching medium stream in the washing zone.

The first quenching medium is phosgene, and the second quenching medium is selected from the group consisting of isocyanate, phosgene, hydrogen chloride, an inert carrier gas, and any combination thereof.

In some embodiments, the reactant amine stream of Step (a) is preheated to 200° C.˜600° C. by a first pre-heater before being passed into the first vessel; and/or the phosgene stream of Step (a) is preheated to 200° C.˜600° C. by a second pre-heater before being passed into the first vessel.

In some embodiments, the reactant amine stream and/or the phosgene stream of Step (a) exist(s) in a gaseous or atomized form before, during or after being passed into the first vessel.

In some embodiments, the reactant amine stream and the phosgene stream of Step (a) are mixed in a headspace of the first vessel.

In some embodiments, the reactant amine stream and the phosgene stream of Step (a) flow top-down in the reaction zone of the first vessel, and react during this process, to obtain the reaction product mixture comprising isocyanate, hydrogen chloride and unreacted phosgene.

In some embodiments, the reactant amine stream and the phosgene stream of Step (a) have a retention time of not more than 260 seconds in the reaction zone of the first vessel.

In some embodiments, the phosgene stream of Step (a) is stoichiometric excess based on amino groups of the reactant amine stream.

In some embodiments, feed ratio (by mole) of the phosgene stream and the reactant amine stream of Step (a) is from 7:1 to 15:1.

In some embodiments, the phosgene stream and/or the reactant amine stream of Step (a) is/are passed into the first vessel simultaneously or sequentially with an inert carrier gas.

In some embodiments, the inert carrier gas is preheated to 200° C.˜600° C. before being passes into the first vessel. In some embodiments, molar flow rate of the inert carrier gas is 10˜100% of molar flow rate of the reactant amine stream or the phosgene stream.

In some embodiments, the first quenching medium of Step (b) and the second quenching medium of Step (d) are the same. In some embodiments, the first quenching medium of Step (b) and the second quenching medium of Step (d) are different. In some embodiments, the first quenching medium of Step (b) and/or the second quenching medium of Step (d) comprise(s) no organic solvent. In some embodiments, the first quenching medium of Step (b) is liquid phosgene. In some embodiments, the second quenching medium is a liquid. In some embodiments, both the first quenching medium and the second quenching medium are liquid phosgene.

In some embodiments, in Step (b), temperature of the reaction product mixture obtained in Step (a) is reduced rapidly by utilizing latent heat of vaporization of the first quenching medium.

In some embodiments, in Step (d), the flowing direction of hydrogen chloride, unreacted phosgene and uncollected isocyanate of Step (c) and the second quenching medium stream are opposite in the washing zone.

In some embodiments, the washing conditions are configurated to allow the hydrogen chloride and unreacted phosgene (and optionally, inert carrier gas) overflowing from top of the second vessel, and the uncollected isocyanate flowing back to the collecting zone of the second vessel. In some embodiments, the hydrogen chloride and unreacted phosgene (and optionally, inert carrier gas) overflowing from top of the second vessel are cooled to −5˜20° C. by a cooler, and then passed through a pressure control system. In some embodiments, the washing conditions is controlling temperature of the second quenching medium and/or the cooler within a range of 0˜15° C. In some embodiments, the hydrogen chloride overflowing from top of the second vessel is subjected to hydrogen chloride refining after being passed through the pressure control system, to form by-product hydrochloric acid.

In some embodiments, the phosgene overflowing from top of the second vessel is recycled, to form the phosgene stream of Step (a) or the first quenching medium stream of Step (b).

In some embodiments, the isocyanate is a diisocyanate. In some embodiments, the isocyanate is an aliphatic diisocyanate or an aromatic diisocyanate. The isocyanate is selected from the group consisting of methylene diphenyl diisocyanate as a pure isomer or as a mixture of isomers, toluene diisocyanate as a pure isomer or as a mixture of isomers, 2,6-xylyl isocyanate, 1,5-naphthalene diisocyanate, methyl isocyanate, ethyl isocyanate, propyl isocyanate, isopropyl isocyanate, butyl isocyanate, isobutyl isocyanate, t-butyl isocyanate, pentyl isocyanate (e.g., pentamethylene diisocyanate), t-pentyl isocyanate, isopentyl isocyanate, neopentyl isocyanate, hexyl isocyanate (e.g., hexamethylene diisocyanate), cyclopentyl isocyanate, cyclohexyl isocyanate, and phenyl isocyanate (e.g., p-phenylene diisocyanate). In some embodiments, the isocyanate is PDI, HDI, IPDI or HTDI.

In some embodiments, the reactant amine has a structural formula of R(NH₂)ₙ, wherein n is 1, 2 or 3, and R is an aliphatic or aromatic hydrocarbyl group. In some embodiments, n is 2, and R is an aliphatic hydrocarbyl group. In some embodiments, n is 2, and R is an aliphatic hydrocarbyl group having 2-10 carbon atoms. In some embodiments, n is 2, and R is a linear or cyclic aliphatic hydrocarbyl group having 3-10 carbon atoms. In some embodiments, the reactant amine exists in a free form. In some embodiments, the reactant amine exists as an amine salt. In some embodiments, the amine salt is selected from the group consisting of a hydrochloride, a sulfate, a bisulfate, a nitrate, and a carbonate.

In some embodiments, the reactant amine is one or more selected from the group consisting of ethyl amine, butyl amine, pentamethylene diamine, hexamethylene diamine, 1,4-diamino butane, 1,8-diamino octane, aniline, p-phenylene diamine, m-xylylene diamine, toluene diamine, 1,5-naphthalene diamine, diphenylmethane diamine, dicyclohexylmethane diamine, m-cyclohexyldimethylene diamine, isophorone diamine, and trans-1,4-cyclohexane diamine. In some embodiments, the reactant amine is selected from the group consisting of PDA, PDA hydrochloride, HDA, HDA hydrochloride, IPDA, IPDA hydrochloride, HTDA and HTDA hydrochloride, the first quenching medium is liquid phosgene, and the second quenching medium is selected from the group consisting of liquid PDI, liquid HDI, liquid phosgene, liquid nitrogen, liquid carbon dioxide or liquid hydrogen chloride. In some embodiments, both the first quenching medium and the second quenching medium are liquid phosgene. In some embodiments, when the first quenching medium is phosgene, the ratio of flow rates of the phosgene as the first quenching medium to the phosgene stream of Step (a) is from 0.7:1 to 1.2:1.

According to the method for preparing an isocyanate provided in the present application, the use of a solvent (particularly an organic solvent) can be avoided during the preparation process, the energy consumption is significantly reduced, the preparation process of isocyanate is simplified, and the equipment investment is lowered, thereby saving the cost for large-scale preparation of isocyanate and alleviating the environmental pollution.

The summary of the present application is described above, and details may be simplified, generalized and omitted. Therefore, it is to be appreciated by those skilled in the art that this section is merely illustrative and not intended to limit the scope of the present application in any way. This summary is not intended to define key features or essential features of the claimed subject matter, nor is it intended to be used as an aid in defining the scope of the claimed subject matter.

### BRIEF DESCRIPTION OF THE DRAWINGS

The above-mentioned and other features of the present application will be more fully and clearly understood from the following description and appended claims in conjunction with the accompanying drawings. It can be understood that these drawings only depict several embodiments of the disclosure of the present application, and therefore should not be considered as a limitation on the scope of the disclosure of the present application. The disclosure of the present application will be explained more clearly and in detail with reference to the accompanying drawings.

FIG. 1 schematically shows a flow chart of a method for preparing an isocyanate according to an embodiment of the present application. In the drawing, E01 is a first pre-heater; E02 is a second pre-heater; E03 is a cooler; 101 is a reaction zone of the first vessel; 102 is a quenching zone of the first vessel; 201 is a collecting zone of the second vessel; 202 is a washing zone of the second vessel; P01 is a pump.

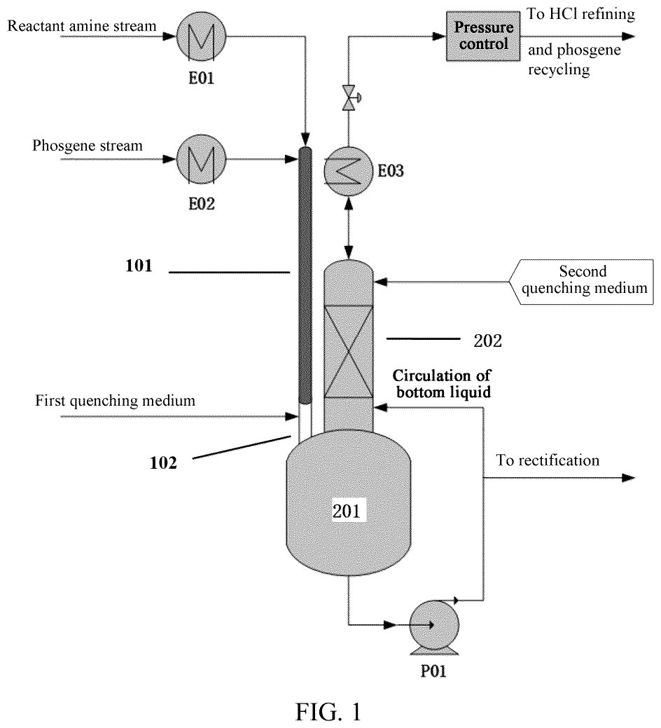

### DETAILED DESCRIPTION

The illustrative embodiments described in the detailed description, drawings and claims are not intended to be limiting. Other embodiments may be adopted and other changes may be made without departing from the spirit or scope of the subject matter of the present application. It can be understood that various aspects of the disclosure of the present application generally described in the present application and illustrated in the attached drawings can be configured, substituted, combined and designed in many different forms, and all of these expressly constitute a part of the present application.

In one aspect, the present application provides a method for preparing an isocyanate, which includes the following steps:
(a) providing a reactant amine stream and a phosgene stream, being passed through and reacting in a reaction zone of a first vessel at a temperature of 200° C.˜600° C., to obtain a reaction product mixture comprising isocyanate, hydrogen chloride and unreacted phosgene;
(b) contacting the reaction product mixture obtained in Step (a) with a first quenching medium stream introduced into a quenching zone of the first vessel, and introducing, via an inlet of a second vessel connected to the quenching zone of the first vessel, the reaction product mixture into the second vessel;
(c) the second vessel of Step (b) comprises a collecting zone and a washing zone, collecting isocyanate in the collecting zone, and passing the hydrogen chloride, unreacted phosgene, and uncollected isocyanate through the washing zone; and
(d) introducing a second quenching medium stream into the washing zone of the second vessel of Step (c), and contacting the hydrogen chloride, unreacted phosgene and uncollected isocyanate of Step (c) with the second quenching medium stream in the washing zone.

The first quenching medium and the second quenching medium are independently selected from the group consisting of isocyanate, phosgene, hydrogen chloride, an inert carrier gas, and any combination thereof, and the ratio of flow rates of the first quenching medium to the phosgene stream of Step (a) is from 0.4:1 to 2:1.

In another aspect, the present application provides a method for preparing an isocyanate, which includes the following steps:
(a) providing a reactant amine stream and a phosgene stream, being passed through and reacting in a reaction zone of a first vessel at a temperature of 200° C.˜600° C., to obtain a reaction product mixture comprising isocyanate, hydrogen chloride and unreacted phosgene;
(b) contacting the reaction product mixture obtained in Step (a) with a first quenching medium stream introduced into a quenching zone of the first vessel, and introducing, via an inlet of a second vessel connected to the quenching zone of the first vessel, the reaction product mixture into the second vessel;
(c) the second vessel of Step (b) comprises a collecting zone and a washing zone, collecting isocyanate in the collecting zone, and passing the hydrogen chloride, unreacted phosgene, and uncollected isocyanate through the washing zone; and
(d) introducing a second quenching medium stream into the washing zone of the second vessel of Step (c), and contacting the hydrogen chloride, unreacted phosgene and uncollected isocyanate of Step (c) with the second quenching medium stream in the washing zone.

The first quenching medium is phosgene, and the second quenching medium is selected from the group consisting of isocyanate, phosgene, hydrogen chloride, an inert carrier gas, and any combination thereof.

In the present application, “isocyanates” refer to a class of compounds containing one or more (for example, two, three, four, five, six, seven, eight, min, ten or more) isocyanate groups (R—N═C═O), including aliphatic isocyanates, aromatic isocyanates, unsaturated isocyanates, halogenated isocyanates, thioisocyanates, phosphorus-containing isocyanates, inorganic isocyanates and blocked isocyanates. In some embodiments, the isocyanate in the present application is a diisocyanate. In some embodiments, the isocyanate in the present application is an aliphatic diisocyanate or an aromatic diisocyanate. In some embodiments, the isocyanate in the present application includes an aromatic isocyanate and an aliphatic isocyanate. For example, aromatic isocyanates include methylene diphenyl diisocyanate as a pure isomer or as a mixture of isomers, toluene diisocyanate as a pure isomer or as a mixture of isomers, 2,6-xylyl isocyanate, 1,5-naphthalene diisocyanate, and the like. Aliphatic isocyanates include methyl isocyanate, ethyl isocyanate, propyl isocyanate, isopropyl isocyanate, butyl isocyanate, isobutyl isocyanate, t-butyl isocyanate, pentyl isocyanate, t-pentyl isocyanate, isopentyl isocyanate, neopentyl isocyanate, hexyl isocyanate, cyclopentyl isocyanate, cyclohexyl isocyanate, phenyl isocyanate, and the like. In some embodiments, the isocyanate in the present application is selected from the group consisting of pentamethylene diisocyanate, hexamethylene diisocyanate, p-phenylene diisocyanate, and toluene diisocyanate. In some embodiments, the isocyanate in the present application is pentamethylene diisocyanate (PDI), hexamethylene diisocyanate (HDI), isophorone diisocyanate (IPDI) or methylcyclohexane diisocyanate (HTDI).

Step (a), Step (b), Step (c) and Step (d) of the method for preparing an isocyanate according to the present application are described in detail below.

### 1. Step (a)

In Step (a) in the present application, a reactant amine stream and a phosgene stream are provided, and then passed through and reacted in a reaction zone of a first vessel at a temperature of 200° C.-600° C., to obtain a reaction product mixture containing isocyanate, hydrogen chloride and unreacted phosgene.

In the present application, the “reactant amine” refers to a compound having an amino (—NH₂) group as a starting material for preparing the isocyanate. For example, in some embodiments, the reactant amine has a structural formula of R(NH₂)ₙ, wherein n is 1, 2 or 3, and R is an aliphatic or aromatic hydrocarbyl group. In some embodiments, n is 2, and R is an aliphatic hydrocarbyl group. In some embodiments, n is 2, and R is an aliphatic, alicyclic or aromatic hydrocarbyl group having 2-10 carbon atoms (for example, 2, 3, 4, 5, 6, 7, 8, 9, or 10 carbon atoms). In some embodiments, n is 2, and R is a linear or cyclic aliphatic hydrocarbyl group having 3-10 carbon atoms (for example, 3, 4, 5, 6, 7, 8, 9, or 10 carbon atoms).

In some embodiments, the reactant amine is a primary amine, that is, having an NH₂ group. In some embodiments, the reactant amine is a diamine, that is, having two NH₂ groups. In some embodiments, the reactant amine is one or more selected from the group consisting of ethyl amine, butyl amine, pentamethylene diamine, hexamethylene diamine, 1,4-diamino butane, 1,8-diamino octane, aniline, p-phenylene diamine, m-xylylene diamine, toluene diamine, 1,5-naphthalene diamine, diphenylmethane diamine, dicyclohexylmethane diamine, m-cyclohexyldimethylene diamine, isophorone diamine, methyl cyclohexane diamine and trans-1,4-cyclohexane diamine. In some embodiments, the reactant amine is selected from the group consisting of pentamethylene diamine (e.g., 1,5-pentamethylene diamine), hexamethylene diamine (e.g., 1,6-hexamethylene diamine), p-phenylene diamine, isophorone diamine, methyl cyclohexane diamine, and toluene diamine.

In some embodiments, the reactant amine exists in a free form. The term “free form” refers to an amine compound in a non-salt form. A free amine compound can be different from their various salt forms in some physical and/or chemical properties, for example, in their solubility in a polar solvent. The free amine compound can also be the same as or similar to their various salt forms in some physical and/or chemical properties.

In some embodiments, the reactant amine exists as an amine salt. In some embodiments, the amine salt is selected from the group consisting of a hydrochloride, a sulfate, a bisulfate, a nitrate, and a carbonate.

In some embodiments, the reactant amine is one or more selected from the group consisting of pentamethylene diamine (PDA), PDA hydrochloride, hexamethylene diamine (HDA), HDA hydrochloride, isophorone diamine (IPDA), IPDA hydrochloride, methylcyclohexane diamine (HTDA) and HTDA hydrochloride.

The isocyanate is usually prepared by the reaction of an amine with phosgene. In the preparation of isocyanate, temperature of reaction with phosgene varies with the types of the reactant amine used. In general, the reactant amine is reacted with phosgene at a temperature of 200° C.˜600° C. (for example, 250° C., 300° C., 310° C., 320° C., 330° C., 340° C., 350° C., 360° C., 370° C., 380° C., 390° C., 400° C., 450° C., 500° C., 550° C. or any temperature within a range between any two of the above values) to produce the isocyanate.

The reaction of the amine with phosgene often takes place stepwise. Firstly, carbamoyl chloride (RNHCOCl) is formed with the amine and phosgene at a low temperature, and then it is converted into a corresponding isocyanate (R—N═C═O) at elevated temperature, in which hydrogen chloride is eliminated in both steps.

For example, pentamethylene diamine and phosgene are used as starting materials, and the main reactions in the reaction zone of the first vessel are as follows:

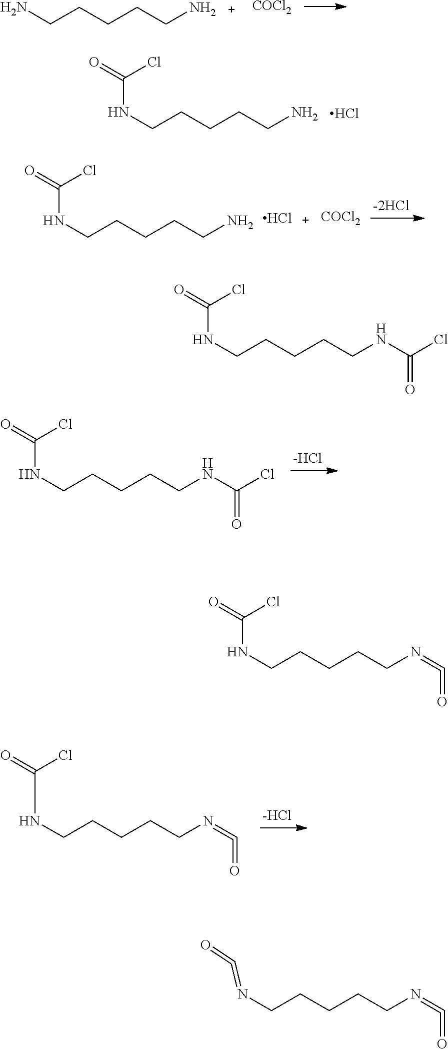

In some embodiments, the reactant amine stream of Step (a) exists in a gaseous or atomized form before, during or after being passed into the first vessel. The vaporization of the reactant amine can be carried out in a known evaporation apparatus. Generally, evaporation may lead to the decomposition of the reactant amine. To reduce the decomposition of the reactant amine, it is usually advantageously to be evaporated, for example, at a lower temperature under a lower pressure (for example, under an absolute pressure of 75˜85 kPa). In some embodiments, the phosgene stream of Step (a) exists in a gaseous or atomized form before, during or after being passed into the first vessel. In Step (a), the reactant amine stream can be passed to the first vessel in a single substream containing the reactant amine, or in multiple substreams (for example, 2, 3, 4, 5 or more) containing the reactant amine. Similarly, in Step (a), the phosgene stream can be passed into the first vessel in a single substream containing phosgene, or in multiple substreams (for example, 2, 3, 4, 5 or more) containing phosgene. When the reactant amine stream (or phosgene stream) of Step (a) is passed into the first vessel in multiple substreams containing the reactant amine (or phosgene), the multiple substreams can be passed into the first vessel at the same position or at different positions.

In the process of preparing the isocyanate, a large amount of excess phosgene is generally required to be added, because when the concentration of phosgene is insufficient, the formed isocyanate will react with excess amine form urea or other high-viscosity solid by-products. Therefore, to prevent the formation of by-products, it is preferable to provide excess phosgene. For example, in some embodiments, the phosgene stream of Step (a) is stoichiometric excess based on amino groups of the reactant amine stream. For example, molar ratio of phosgene to the amino group of the reactant amine is usually 1.1:1-30:1 (for example, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, 11:1, 15:1, 20:1, 25:1, 30:1 and a range between any two of the above values). In some embodiments, in the first vessel, phosgene is used in a stoichiometrically excess amount based on amino groups of the reactant amine, which is 0% to 250% (for example, 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, or 250%) more than the theoretical value. When the reactant amine stream (and/or phosgene stream) of Step (a) is passed into the first vessel in multiple substreams containing the reactant amine (and/or phosgene), the total phosgene stream produced by the sum of the multiple substreams containing phosgene is stoichiometric excess based on amino groups of the total reactant amine stream produced by the sum of the multiple streams containing the reactant amine.

In some embodiments, feed ratio (by mole) of the phosgene stream and the reactant amine stream of Step (a) is from 7:1 to 15:1 (for example, 8:1, 9:1, 10:1, 11:1, 12:1, 13:1, 14:1 or any value between any two ratios above). Further, the feed ratio (by mole) of the phosgene stream and the reactant amine stream of Step (a) is from 10:1 to 14:1.

In Step (a), the phosgene contained in the phosgene stream can be fresh phosgene or recycled phosgene. The term “fresh phosgene” refers to a stream containing phosgene that is not recycled from a phosgenation process and has not undergone any reaction stage involving a phosgenation reaction after the phosgene is usually synthesized with chlorine and carbon monoxide. The term “recycled phosgene” refers to a stream containing phosgene generated by collecting a tail gas of a reaction process for preparing an isocyanate by phosgenation. As mentioned above, in the process of preparing an isocyanate by a gas-phase method, use of excess phosgene is generally required, so there will be a large amount of phosgene in the reaction tail gas. Recycling of the phosgene in the tail gas can reduce the production cost.

In some embodiments, the reactant amine stream of Step (a) is preheated by a first pre-heater to a temperature required for the reaction of the reactant amine and phosgene to produce the isocyanate, for example, 200° C.˜600° C. (e.g., 250° C., 300° C., 310° C., 320° C., 330° C., 340° C., 350° C., 360° C., 370° C., 380° C., 390° C., 400° C., 450° C., 500° C., 550° C. or a range between any two of the above values) before being passed into the first vessel. In some embodiments, the phosgene stream of Step (a) is preheated by a second pre-heater to a temperature required for the reaction of the reactant amine and phosgene to produce the isocyanate, for example, 200° C.˜600° C. (e.g., 250° C., 300° C., 310° C., 320° C., 330° C., 340° C., 350° C., 360° C., 370° C., 380° C., 390° C., 400° C., 450° C., 500° C., 550° C. or a range between any two of the above values) before being passed into the first vessel. In some embodiments, the reactant amine stream and the phosgene stream of Step (a) are mixed in a mixing apparatus and then preheated by a pre-heater, for example, to a temperature required for the reaction of the reactant amine and phosgene to produce the isocyanate, for example, 200° C.˜600° C. (e.g., 250° C., 300° C., 310° C., 320° C., 330° C., 340° C., 350° C., 360° C., 370° C., 380° C., 390° C., 400° C., 450° C., 500° C., 550° C. or a range between any two of the above values) before being passed into the first vessel. The reactant amine and/or phosgene can be preheated by direct or indirect heating by steam heating, by an electric heater or by fuel combustion.

In some embodiments, the reactant amine stream and the phosgene stream of Step (a) are mixed in a headspace of the first vessel.

In some embodiments, the reactant amine stream and the phosgene stream of Step (a) are reacted in the reaction zone of the first vessel under an absolute pressure of 0.05˜0.2 MPa (for example, 0.06 Mpa, 0.07 Mpa, 0.08 Mpa, 0.09 Mpa, 0.1 Mpa, 0.11 Mpa, 0.12 Mpa, 0.13 Mpa, 0.14 Mpa, 0.15 Mpa, 0.16 Mpa, 0.17 Mpa, 0.18 Mpa, 0.19 Mpa or a range between any two of the above values). The reaction is preferably carried out under an absolute pressure of 0.05˜0.12 Mpa, and more preferably 0.08˜0.1 Mpa.

In some embodiments, the reactant amine stream and the phosgene stream of Step (a) have a retention time of no more than 260 seconds in the reaction zone of the first vessel, for example, no more than 250 seconds, no more than 240 seconds, no more than 230 seconds, no more than 220 seconds, no more than 210 seconds, no more than 200 seconds, no more than 190 seconds, no more than 180 seconds, no more than 170 seconds, no more than 160 seconds, no more than 150 seconds, no more than 140 seconds, no more than 130 seconds, no more than 120 seconds, no more than 110 seconds, no more than 100 seconds, etc. Preferably, the reactant amine stream and the phosgene stream of Step (a) have a retention time of no more than 10 seconds in the reaction zone of the first vessel, for example, no more than 1 second, 2 seconds, 3 seconds, 3.5 seconds, 4 seconds, 4.5 seconds, 5 seconds, 5.5 seconds, 6 seconds, 6.5 seconds, 7 seconds, 7.5 seconds, 8 seconds, 8.5 seconds, or 9 seconds. Without being bound by any theory, it is considered that it is preferable to maintain a short reaction time of the reactant amine and phosgene, to avoid the formation of by-products as much as possible. The residence time of the reactant amine stream and the phosgene stream in the reaction zone of the first vessel can be controlled in many ways. For example, the residence time in the reaction zone of the first vessel can be controlled by controlling the flow rate of the reactant amine stream and/or the phosgene stream by a flow control device. For example, the reaction residence time in the first vessel is shortened or prolonged by increasing or decreasing the flow rate of an inert medium in the amine stream and/or the phosgene stream. For example, as the flow rate of the reactant amine stream and/or the phosgene stream increases, the residence time in the reaction zone of the first vessel decreases. In addition, to ensure that the reactant amine stream and the phosgene stream can complete the reaction in a time as short as possible, it is preferable to mix them as evenly as possible, for example, by a suitable mixing device (for example, a mixing unit or mixing zone with a dynamic or static mixing element or nozzle).

In some embodiments, the flow rates of the reactant amine and the phosgene are adjusted respectively by an amine metering pump and a phosgene metering pump of the first vessel, such that the reactant amine and the phosgene can be added into the first vessel at a constant rate for reaction. For example, the reactant amine (in mole) is introduced into the first vessel at a constant rate of 1˜5 mol/h (for example, 1 mol/h, 2 mol/h, 3 mol/h, 4 mol/h, 5 mol/h or any value within a range between any two of the above values), and the phosgene is introduced into the first vessel at a constant rate of 7˜60 mol/h (for example, 7 mol/h, 10 mol/h, 20 mol/h, 30 mol/h, 40 mol/h, 50 mol/h, 60 mol/h or any value within a range between any two of the above values). Without being bound by any theory, it is considered that it is preferable to keep a constant flow rate of the reactant amine and the phosgene, such that the feeding amount can be accurately controlled, to ensure the continuous production of the isocyanate and ensure the yield of the isocyanate.

In some embodiments, the reactant amine stream and the phosgene stream of Step (a) flow top-down in the reaction zone of the first vessel, and react during this process, to obtain the reaction product mixture comprising isocyanate, hydrogen chloride and unreacted phosgene.

The phosgene stream and/or the reactant amine stream of Step (a) can also be passed into the first vessel simultaneously or sequentially with an inert carrier gas. The inert carrier gas can promote the vaporization of the reactant amine and can enable a more suitable dispersion effect. The inert carrier gas is a medium that exists in a gaseous state in the reaction vessel at the reaction temperature and that basically does not react with the reactants or compounds present in the reaction process or is stable under the reaction conditions. Exemplary inert carrier gas includes nitrogen, carbon dioxide, carbon monoxide, helium or argon. In some embodiments, the inert carrier gas is preheated to 200° C.˜600° C. (e.g., 250° C., 300° C., 310° C., 320° C., 330° C., 340° C., 350° C., 360° C., 370° C., 380° C., 390° C., 400° C., 450° C., 500° C., 550° C. or a range between any two of the above values) before being passed into the first vessel. In some embodiments, the inert carrier gas (e.g., nitrogen) is passed into the first vessel at a constant rate of 2˜7 L/h (for example, 2 L/h, 3 L/h, 3.5 L/h, 4 L/h, 4.48 L/h, 4.5 L/h, 5 L/h, 5.5 L/h, 6 L/h, 6.5 L/h, 7 L/h or any value within a range between any two of the above values). In some embodiments, the molar flow rate of the inert carrier gas is 10-100% of that of reactant amine or phosgene (for example, 15%, 20%, 25%, 30%, 35%, 40%, 50%, 60%, 70%, 80%, 90%, 95%, or any value within a range between any two of the above values). When the flow rate of the reactant amine stream is very low, the flow rate of the inert carrier gas is preferably increased to ensure a proper linear speed.

The first vessel can be any type of conventional reaction vessels known in the prior art and suitable for non-catalytic homogeneous gas reaction, preferably suitable for continuous non-catalytic homogeneous gas reaction and tolerant to moderate pressure required, such as those disclosed in EP289840B1, and EP593334B1, etc. The first vessel can be made of a metal (for example, steel, silver, or copper), glass, ceramic or enamel. Preferably, a steel reactor is used. The wall of the first vessel can be smooth or contoured (e.g. grooved or wrinkled).

In some embodiments, the first vessel can be a tubular reactor. For example, the upper part of the tubular reactor is the reaction zone and the lower part is the quenching zone. In addition to the tubular reactor, a reaction space that is basically cuboid or cubic, such as a plate reactor, can also be used, and reactors with any other desired cross-sectional shapes can also be used.

### 2. Step (b)

In Step (b) in the present application, the reaction product mixture obtained in Step (a) is contacted with a first quenching medium stream introduced into a quenching zone of the first vessel, and introduced, via an inlet of a second vessel connected to the quenching zone of the first vessel, into the second vessel.

To reduce or prevent the formation of by-products and to suppress the decomposition of the formed isocyanate, the reaction product mixture is brought into contact with the first quenching medium stream introduced into the quenching zone of the first vessel immediately after the reaction, to cool the reaction product mixture. Preferred first quenching medium is a liquid quenching medium, and the first quenching medium absorbs heat through evaporation and accordingly leads to the rapid cooling of the reaction product mixture. In some embodiments, the first queuing medium exists in the form of a fine spray, so as to realize the rapid cooling of the reaction product mixture. For example, the reaction product mixture is cooled by the first quenching medium to a temperature between 110˜150° C., such as 110° C., 120° C., 130° C., 140° C., 150° C. or a range between any two of the above values.

The “quenching zone of the first vessel” in the present application can also be another vessel separated from the first vessel, which has similar functions or effects, that is, to provide a space for the contact between the reaction product mixture and the first quenching medium stream, so as to cool the reaction product mixture and capture the target product isocyanate.

In the art, the commonly used quenching medium includes a solvent, an isocyanate or a mixture of an isocyanate and a solvent. The first quenching medium in the present application is selected from the group consisting of an isocyanate, phosgene, hydrogen chloride, an inert carrier gas, and any combination thereof. The yield of the product can be optimized by adjusting the flow rates of the first quenching medium and the phosgene stream of Step (a). The ratio of the flow rates of the first quenching medium to the phosgene stream of Step (a) can be adjusted to be between 0.4:1˜2:1 (for example, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, 2:1 or any value within a range between any two of the above ratios). For example, when a solvent (e.g., 1,2-dichlorobenzene) is used as the first quenching medium, the ratio of the flow rates of the solvent (e.g., 1,2-dichlorobenzene) to the phosgene stream of Step (a) is 0.4:1-0.6:1 (e.g., 0.54:1); when an isocyanate (e.g., PDI) is used as the first quenching medium, the ratio of the flow rates of the isocyanate (e.g., PDI, or HDI) to the phosgene stream of Step (a) is 0.5:1-0.8:1 (e.g., 0.68:1); when an inert carrier gas (e.g., nitrogen, or carbon dioxide) is used as the first quenching medium, the ratio of the flow rate of the inert carrier gas (e.g., nitrogen, or carbon dioxide) to the phosgene stream of Step (a) is 1.2:1-1.8:1 (e.g., 1.4:1, or 1.6:1); when hydrogen chloride is used as the first quenching medium, the ratio of the flow rates of hydrogen chloride to the phosgene stream of Step (a) is 1.2:1-1.6:1 (for example, 1.4:1).

The inventors of the present application also unexpectedly found that by using phosgene solely as the first quenching medium to cool the reaction product mixture obtained in Step (a), the use of an organic solvent in the whole reaction system can be avoided, and the problem of inlet blockage caused by solid adhesion can also be avoided, so that no solvent recovery, rectification and recycled refining procedures are required in the whole process, and the preparation process is simple, low in energy consumption and low in cost. Moreover, due to the shortened high-temperature refining process, the high-temperature residence time of the reaction product isocyanate is greatly reduced, the self-polymerization reaction is alleviated, and the yield of the product is high.

In some embodiments, the first quenching medium is not or does not contain a solvent. In some embodiments, the first quenching medium is not or does not contain an organic solvent (for example, chlorobenzene, toluene, hexane, tetrahydrofuran, chloronaphthalene, and the like). In some embodiments, the first quenching medium is not or does not contain an isocyanate.

In some embodiments, in Step (b), temperature of the reaction product mixture obtained in Step (a) is reduced rapidly by utilizing the latent heat of vaporization of the first quenching medium. For example, temperature of the reaction product mixture obtained in Step (a) is reduced rapidly by the first quenching medium, for example, by at least 200° C./second. Without being bound by any theory, it is considered that it is preferable to rapidly reduce temperature of the reaction product mixture obtained in Step (a), to ensure the purity of the target product. If temperature is not lowered in a time as short as possible, the reaction product mixture obtained in Step (a) may undergo polymerization to produce impurities. Temperature of the reaction product mixture can be rapidly reduced in many ways, for example, by increasing the flow rate of the first quenching medium, reducing the initial temperature of the first quenching medium, improving the spray dispersion effect of the first quenching medium to increase the heat exchange rate, etc.

In some embodiments, the contact time of the reaction product mixture obtained in Step (a) with the first quenching medium stream in the quenching zone of the first vessel is no more than 1 second, for example, no more than 0.9 seconds, no more than 0.8 seconds, no more than 0.7 seconds, no more than 0.6 seconds, no more than 0.5 seconds, no more than 0.4 seconds, no more than 0.3 seconds, no more than 0.2 seconds, or no more than 0.1 seconds. Preferably, the contact time of the reaction product mixture obtained in Step (a) with the first quenching medium stream in the quenching zone of the first vessel is between 0.2 and 0.5 seconds.

In some embodiments, Step (b) is carried out under an absolute pressure of 0.05˜0.2 Mpa (for example, 0.06 Mpa, 0.07 Mpa, 0.08 Mpa, 0.09 Mpa, 0.1 Mpa, 0.11 Mpa, 0.12 Mpa, 0.13 Mpa, 0.14 Mpa, 0.15 Mpa, 0.16 Mpa, 0.17 Mpa, 0.18 Mpa, 0.19 Mpa or a range between any two of the above values), preferably 0.05˜0.12 Mpa, and further preferably 0.08˜0.1 Mpa.

In some embodiments, the first quenching medium is phosgene. In some embodiments, the first quenching medium is liquid phosgene. In some embodiments, the first quenching medium is pressurized liquid phosgene. Without being bound by any theory, it is considered that it is particularly advantageous to use pressurized liquid phosgene (for example, the pressure is 0.5˜2 MPa). Because of the high ejection pressure of pressurized phosgene and the continuous boiling of some liquid phosgene on the inner wall surface, a layer of gas cushion film is formed, and the polymer cannot adhere to the inner wall, so the solid precipitation and the blockage of the reaction pipe are avoided. The pressure can be increased by increasing the flow rate of phosgene. For example, the flow rate of the phosgene as the first quenching medium is adjusted to 7˜12 mol/h (for example, 7 mol/h, 8 mol/h, 9 mol/h, 10 mol/h, 11 mol/h, 12 mol/h or any value within a range between any two of the above values). In some embodiments, the first quenching medium is fresh phosgene. In some embodiments, the first quenching medium is recycled phosgene.

In some embodiments, when the first quenching medium is phosgene, the ratio of the flow rate of the phosgene as the first quenching medium to the phosgene stream of Step (a) is from 0.7:1 to 1.2:1 (e.g., 0.7:1, 0.8:1, 0.9:1, 1:1, 1.1:1, 1.2:1 or any ratio within a range between any two of the above ratios).

### 3. Step (c)

The second vessel of Step (b) comprises a collecting zone and a washing zone. In Step (c) in the present application, the isocyanate is collected in the collecting zone of the second vessel, and the hydrogen chloride, unreacted phosgene, and uncollected isocyanate (and optionally the inert carrier gas) are passed through the washing zone of the second vessel.

As described above, in Step (b), the reaction product mixture is cooled to a temperature between 110˜150° C. (for example, 110° C., 120° C., 130° C., 140° C., 150° C. or a range between any two of the above values) by the first quenching medium, whereby the target product isocyanate is liquefied and collected in the collecting zone of the second vessel. However, this temperature cannot enable the hydrogen chloride and unreacted phosgene in the reaction product mixture to be liquefied. Therefore, the hydrogen chloride, unreacted phosgene, and a small amount of non-liquefied isocyanate are still in the form of a gas and fed into the washing zone of the second vessel.

The collecting zone and the washing zone of the second vessel can be deployed in any pattern. Preferably, the collecting zone of the second vessel is located at a lower part of the second vessel, and the washing zone is located at an upper part of the second vessel. In some embodiments, the washing zone of the second vessel is a washing tower with at least one separator stage. In some embodiments, isocyanate collected in the collecting zone of the second vessel is pumped to the washing zone of the second vessel, thus realizing the circulation of the bottom liquid. The circulation of the bottom liquid has at least the following two advantages: being able to replace stirring to realize the disturbance, dispersion and homogenization of the liquid in the second vessel; allowing a stable output flow to the outside, thus making the operation of a following rectification tower more smoothly.

### 4. Step (d)

In Step (d) of the present application, a second quenching medium stream is introduced into the washing zone of the second vessel of Step (c), and the hydrogen chloride, unreacted phosgene and uncollected isocyanate (and optionally, the inert carrier gas) of Step (c) are brought into contact with the second quenching medium stream in the washing zone.

In some embodiments, the second quenching medium of Step (d) is the same as the first quenching medium of Step (b), that is, both are phosgene. In some embodiments, the second quenching medium of Step (d) is different from the first quenching medium of Step (b). For example, the second quenching medium is an isocyanate, a solvent or a mixture thereof. In some embodiments, the second quenching medium is not or does not contain a solvent. In some embodiments, the second quenching medium is not or does not contain an organic solvent (for example, chlorobenzene, toluene, hexane, tetrahydrofuran, chloronaphthalene, and the like). In some embodiments, the second quenching medium is not or does not contain an isocyanate. In some embodiments, the second quenching medium is a liquid. In some embodiments, the second quenching medium is selected from the group consisting of liquid PDI, liquid HDI, liquid phosgene, liquid nitrogen, liquid carbon dioxide or liquid hydrogen chloride. In some embodiments, the second quenching medium is liquid phosgene. In some embodiments, both the first quenching medium and the second quenching medium are liquid phosgene.

In some embodiments, the flowing directions of the hydrogen chloride, unreacted phosgene and uncollected isocyanate (and optionally the inert carrier gas) of Step (c) and the second quenching medium stream are opposite in the washing zone. For example, the hydrogen chloride, unreacted phosgene and uncollected isocyanate (and optionally the inert carrier gas) flow bottom-up, and the second quenching medium flows top-down, so that they are brought into full contact, whereby the uncollected isocyanate of Step (c) is condensed as much as possible from the gas-phase stream. After the uncollected isocyanate of Step (c) is treated in Step (d), the isocyanate condensed into a liquid is collected in the collecting zone of the second vessel.

In Step (d), the washing conditions are controlled such that the hydrogen chloride and unreacted phosgene (and optionally, inert carrier gas) overflow from the top of the second vessel, and the uncollected isocyanate is flowed back to the collecting zone of the second vessel. In this way, the yield of the target product isocyanate is maximized. For example, the hydrogen chloride and unreacted phosgene (and optionally, inert carrier gas) overflowing from the top of the second vessel are cooled to −5˜20° C. (for example, 0° C., 5° C., 10° C., 15° C., 18° C., 19° C. or any temperature within a range between any two of the above values) by a cooler, and then passed through a pressure control system. Preferably, by controlling temperature of the second quenching medium and/or the cooler in a properly low range (for example, 0˜15° C., for example, 1° C., 2° C., 3° C., 4° C., 5° C., 6° C., 7° C., 8° C., 9° C., 10° C., 11° C., 12° C., 13° C., 14° C. or any temperature within a range between any two of the above values), the uncollected isocyanate of Step (c) can be completely condensed and flowed back, while the hydrogen chloride and unreacted phosgene (and optionally, the inert carrier gas) are not condensed and overflow from the top of the second vessel.

In some embodiments, the hydrogen chloride overflowing from the top of the second vessel is subjected to refining of hydrogen chloride after being passed through the pressure control system, to form the by-product hydrochloric acid. In some embodiments, the phosgene overflowing from the top of the second vessel is recycled, to form the phosgene stream of Step (a) or the first quenching medium stream of Step (b).

In some embodiments, the method for preparing an isocyanate according to the present application further includes a Step (e) in which the isocyanate collected in the collecting zone of the second vessel is transferred into a purification device for rectification, to obtain a purified isocyanate. To be clear, Step (e) is not an essential step in the method of the present application for preparing an isocyanate, and the purity of isocyanate obtained by Steps (b) and (d) is high enough, for example, 90% or higher.

According to the method for preparing an isocyanate provided in the present invention, the use of an organic solvent can be avoided, the whole process is solvent free, and recycling use of phosgene is achieved. Compared with the traditional method, the production cost is greatly reduced, the yield of the isocyanate is improved, and the environmental pollution caused by the organic solvent is also significantly reduced. In addition, the rate of conversion to the isocyanate prepared by the method of the present invention can be up to 100%, and the selectivity can be up to 96% or higher. “Conversion rate” refers to the rate of the amount of a reactant (for example, reactant amine) detected after the reaction and before purification by rectification to the amount of such reactant before the reaction. “Selectivity” refers to the rate of the actual target product determined by chromatographic analysis after the reaction and before purification by rectification to 100% theoretical value.

### EXAMPLES

To fully understand the present invention, the following examples are shown. It should be understood that these examples are provided merely for illustrative purposes and are not to be construed as limiting in any way.

Some abbreviations of nouns mentioned in the examples are shown in Table 1.

TABLE 1
Abbreviations of nouns

| Abbreviation | Full name |
|---|---|
| PDI | Pentamethylene diisocyanate |
| PDA | Pentamethylene diamine |
| HDI | Hexamethylene diisocyanate |
| HDA | Hexamethylene diamine |
| IPDI | Isophorone diisocyanate |
| IPDA | Isophorone diamine |
| HTDI | Methylcyclohexane diisocyanate |
| HTDA | Methylcyclohexane diamine |

The raw materials, quenching medium, conversion rate and selectivity in the following examples are summarized in Table 2.

TABLE 2
Summary of raw materials, quenching medium, and results in various examples

| Example | | | Conversion | | |
|---|---|---|---|---|---|
| No. | Raw material | Quenching medium | rate | Selectivity | Remark |
| 1-1 | PDA | 1,2-dichlorobenzene | 100% | 97.4% | Synthesis of |
| 1-2 | PDA | Liquid PDI at −10° C. | 100% | 94.5% | PDI |
| 1-3 | PDA | Liquid phosgene | 100% | 99.3% | |
| 1-4 | PDA | Liquid nitrogen | 100% | 94.2% | |
| 1-5 | PDA | Liquid carbon dioxide | 100% | 92.1% | |
| 1-6 | PDA | Liquid hydrogen chloride | 100% | 94.1% | |
| 1-7 | PDA | Liquid phosgene | 100% | 96.0% | |
| | hydrochloride | | | | |
| 1-8 | PDA | Liquid phosgene | 100% | 98.8% | Synthesis of |
| | | | | | PDI |
| | | | | | Phosgene for |
| | | | | | reaction is 2 |
| | | | | | eq more than |
| | | | | | that in |
| | | | | | Examples 1-3 |
| 2-1 | HDA | 1,2-dichlorobenzene | 100% | 94.5% | Synthesis of |
| | | | | | HDI |
| 2-2 | HDA | Liquid HDI at −10° C. | 100% | 93.1% | |
| 2-3 | HDA | Liquid phosgene | 100% | 97.2% | |
| 2-4 | HDA | Liquid nitrogen | 100% | 94.3% | |
| 2-5 | HDA | Liquid carbon dioxide | 100% | 91.1% | |
| 2-6 | HDA | Liquid hydrogen chloride | 100% | 90.1% | |
| 2-7 | HDA | Liquid phosgene | 100% | 95.8% | |
| | hydrochloride | | | | |
| 3-1 | IPDA | Liquid phosgene | 100% | 95.3% | Synthesis of |
| 3-2 | IPDA | Liquid phosgene | 100% | 94.8% | IPDI |
| | hydrochloride | | | | |
| 4-1 | HTDA | Liquid phosgene | 100% | 96.3% | |
| 4-2 | HTDA | Liquid phosgene | 100% | 96.0% | Synthesis of |
| | hydrochloride | | | | HTDI |

The steps and results of each example are described in detail below.

### Example 1: Synthesis of PDI

### Example 1-1: PDA was Used as a Raw Material and 1,2-Dichlorobenzene was Used as a Quenching Medium

Phosgene and an inert carrier gas were preheated to 330° C. Phosgene at 990 g/h (10 eq) and the inert carrier gas (nitrogen) at 4.48 L/h (that is, 20% by mole of PDA) were mixed and first introduced into a phosgenation reactor. After 10 min, PDA preheated to 330° C. was introduced into the phosgenation reactor at a rate of 102 g/h. The two reactant streams were mixed at the inlet of the reactor. The mixed stream flowed top-down through a reaction zone (residence time 3.5 s) of the phosgenation reactor heated to 330° C. Then the mixed gas after reaction was fed to a quenching zone of the phosgenation reactor. Temperature of the mixed gas after reaction was reduced rapidly by utilizing the latent heat of vaporization of the quenching medium 1,2-dichlorobenzene, where the flow rate of the quenching medium 1,2-dichlorobenzene (−10° C., 0.6 MPa) was 800 g/h. The target product was cooled to 130° C. and then collected at the bottom of a reaction absorption vessel (collecting vessel). In this process, 1,2-dichlorobenzene was used to capture and absorb the target product. By weighing and liquid chromatography, the conversion rate of the reaction was 100% and the selectivity was 97.4%.

### Example 1-2: PDA was Used as a Raw Material and Liquid PDI at −10° C. was Used as a Quenching Medium

Phosgene and an inert carrier gas were preheated to 330° C. Phosgene at 990 g/h (10 eq) and the inert carrier gas (nitrogen) at 4.48 L/h (that is, 20% by mole of PDA) were mixed and first introduced into a phosgenation reactor. After 10 min, PDA preheated to 330° C. was introduced into the phosgenation reactor at a rate of 102 g/h. The two reactant streams were mixed at the inlet of the reactor. The stream was then flowed through a reaction zone of the phosgenation reactor heated to 330° C. in 3.5 s (that is, the residence time was 3.5 s). Then the mixed gas after reaction was fed to a quenching zone of the phosgenation reactor. Temperature of the mixed gas after reaction was reduced rapidly by utilizing the latent heat of vaporization of the quenching medium (liquid PDI cooled to −10° C.), where the flow rate of the quenching medium PDI (−10° C., 0.5 MPa) was 1050 g/h. The target product was cooled to 130° C. and then collected at the bottom of a reaction absorption vessel (collecting vessel). In this process, liquid PDI cooled to −10° C. was used to capture and absorb the target product. By weighing and gas chromatography, the conversion rate of the reaction was 100% and the selectivity was 94.5%.

### Example 1-3: PDA was Used as a Raw Material, and Liquid Phosgene was Used as a Quenching Medium

Phosgene and an inert carrier gas were preheated to 330° C. Phosgene at 990 g/h (10 eq) and the inert carrier gas (nitrogen) at 4.48 L/h (that is, 20% by mole of PDA) were mixed and first introduced into a phosgenation reactor. After 10 min, PDA preheated to 330° C. was introduced into the phosgenation reactor at a rate of 102 g/h. The two reactant streams were mixed at the inlet of the reactor. The stream was then flowed through a reaction zone of the phosgenation reactor heated to 330° C. in 3.5 s (that is, the residence time was 3.5 s). Then the mixed gas after reaction was fed to a quenching zone of the phosgenation reactor. Temperature of the mixed gas after reaction was reduced rapidly by utilizing the latent heat of vaporization of the quenching medium (liquid phosgene), where the flow rate of the quenching medium liquid phosgene (−10° C., 0.5 MPa) was 800 g/h. The target product was cooled to 130° C. and then collected at the bottom of a reaction absorption vessel (collecting vessel). In this process, liquid phosgene was used to capture and absorb the target product. By weighing and gas chromatography, the conversion rate of the reaction was 100% and the selectivity was 99.3%.

### Example 1-4: PDA was Used as a Raw Material, and Liquid Nitrogen was Used as a Quenching Medium

Phosgene and an inert carrier gas were preheated to 330° C. Phosgene at 990 g/h (10 eq) and the inert carrier gas (nitrogen) at 4.48 L/h (that is, 20% by mole of PDA) were mixed and first introduced into a phosgenation reactor. After 10 min, PDA preheated to 330° C. was introduced into the phosgenation reactor at a rate of 102 g/h. The two reactant streams were mixed at the inlet of the reactor. The stream was then flowed through a reaction zone of the phosgenation reactor heated to 330° C. in 3.5 s (that is, the residence time was 3.5 s). Then the mixed gas after reaction was fed to a quenching zone of the phosgenation reactor. Temperature of the mixed gas after reaction was reduced rapidly by utilizing the latent heat of vaporization of the quenching medium (liquid nitrogen), where the flow rate of the quenching medium liquid nitrogen (−176° C., 0.5 MPa) was 450 g/h. The target product was cooled to 130° C. and then collected at the bottom of a reaction absorption vessel (collecting vessel). In this process, liquid nitrogen was used to capture and absorb the target product. By weighing and gas chromatography, the conversion rate of the reaction was 100% and the selectivity was 94.2%.

### Example 1-5: PDA was Used as a Raw Material, and Liquid Carbon Dioxide was Used as a Quenching Medium

Phosgene and an inert carrier gas were preheated to 330° C. Phosgene at 990 g/h (10 eq) and the inert carrier gas (nitrogen) at 4.48 L/h (that is, 20% by mole of PDA) were mixed and first introduced into a phosgenation reactor. After 10 min, PDA preheated to 330° C. was introduced into the phosgenation reactor at a rate of 102 g/h. The two reactant streams were mixed at the inlet of the reactor. The stream was then flowed through a reaction zone of the phosgenation reactor heated to 330° C. in 3.5 s (that is, the residence time was 3.5 s). Then the mixed gas after reaction was fed to a quenching zone of the phosgenation reactor. Temperature of the mixed gas after reaction was reduced rapidly by utilizing the latent heat of vaporization of the quenching medium (liquid carbon dioxide), where the flow rate of the quenching medium liquid carbon dioxide (−46° C., 0.7 MPa) was 600 g/h. The target product was cooled to 130° C. and then collected at the bottom of a reaction absorption vessel (collecting vessel). In this process, liquid carbon dioxide was used to capture and absorb the target product. By weighing and gas chromatography, the conversion rate of the reaction was 100% and the selectivity was 92.1%.

### Example 1-6: PDA was Used as a Raw Material, and Liquid Hydrogen Chloride was Used as a Quenching Medium

Phosgene and an inert carrier gas were preheated to 330° C. Phosgene at 990 g/h (10 eq) and the inert carrier gas (nitrogen) at 4.48 L/h (that is, 20% by mole of PDA) were mixed and first introduced into a phosgenation reactor. After 10 min, PDA preheated to 330° C. was introduced into the phosgenation reactor at a rate of 102 g/h. The two reactant streams were mixed at the inlet of the reactor. The stream was then flowed through a reaction zone of the phosgenation reactor heated to 330° C. in 3.5 s (that is, the residence time was 3.5 s). Then the mixed gas after reaction was fed to a quenching zone of the phosgenation reactor. Temperature of the mixed gas after reaction was reduced rapidly by utilizing the latent heat of vaporization of the quenching medium (liquid hydrogen chloride), where the flow rate of the quenching medium liquid hydrogen chloride (−40° C., 0.7 MPa) was 520 g/h. The target product was cooled to 130° C. and then collected at the bottom of a reaction absorption vessel (collecting vessel). In this process, liquid hydrogen chloride was used to capture and absorb the target product. By weighing and gas chromatography, the conversion rate of the reaction was 100% and the selectivity was 94.1%.

### Example 1-7: PDA Hydrochloride was Used as a Raw Material, and Liquid Phosgene was Used as a Quenching Medium

Phosgene and an inert carrier gas were preheated to 330° C. Phosgene at 990 g/h (10 eq) and the inert carrier gas (nitrogen) at 4.48 L/h (that is, 20% by mole of PDA hydrochloride) were mixed and first introduced into a phosgenation reactor. After 10 min, gaseous PDA hydrochloride preheated to 330° C. was introduced into the phosgenation reactor at a rate of 175 g/h. The two reactant streams were mixed at the inlet of the reactor. The stream was then flowed through a reaction zone of the phosgenation reactor heated to 330° C. in 3.5 s (that is, the residence time was 3.5 s). Then the mixed gas after reaction was fed to a quenching zone of the phosgenation reactor. Temperature of the mixed gas after reaction was reduced rapidly by utilizing the latent heat of vaporization of the quenching medium (liquid phosgene), where the flow rate of the quenching medium liquid phosgene (−10° C., 0.5 MPa) was 800 g/h. The target product was cooled to 130° C. and then collected at the bottom of a reaction absorption vessel (collecting vessel). In this process, liquid phosgene was used to capture and absorb the target product. By weighing and gas chromatography, the conversion rate of the reaction was 100% and the selectivity was 96.0%.

### Example 1-8: The Amount of Phosgene was 12 eq, PDA was Used as a Raw Material, and Liquid Phosgene was Used as a Quenching Medium

Phosgene and an inert carrier gas were preheated to 330° C. Phosgene at 1188 g/h (12 eq) and the inert carrier gas (nitrogen) at 4.48 L/h (that is, 20% by mole of PDA) were mixed and first introduced into a phosgenation reactor. After 10 min, PDA preheated to 330° C. was introduced into the phosgenation reactor at a rate of 102 g/h. The two reactant streams were mixed at the inlet of the reactor. The stream was then flowed through a reaction zone of the phosgenation reactor heated to 330° C. in 3.5 s (that is, the residence time was 3.5 s). Then the mixed gas after reaction was fed to a quenching zone of the phosgenation reactor. Temperature of the mixed gas after reaction was reduced rapidly by utilizing the latent heat of vaporization of the quenching medium (liquid phosgene), where the flow rate of the quenching medium liquid phosgene (−10° C., 0.5 MPa) was 850 g/h. The target product was cooled to 130° C. and then collected at the bottom of a reaction absorption vessel (collecting vessel). In this process, liquid phosgene was used to capture and absorb the target product. By weighing and gas chromatography, the conversion rate of the reaction was 100% and the selectivity was 98.8%.

### Example 2 Synthesis of HDI

### Example 2-1: HDA was Used as a Raw Material and Dichlorobenzene was Used as a Quenching Medium

Phosgene and an inert carrier gas were preheated to 340° C. Phosgene at 990 g/h (10 eq) and the inert carrier gas (nitrogen) at 4.48 L/h (that is, 20% by mole of HDA) were mixed and first introduced into a phosgenation reactor. After 10 min, HDA preheated to 340° C. was introduced into the phosgenation reactor at a rate of 116 g/h. The two reactant streams were mixed at the inlet of the reactor. The stream was then flowed through a reaction zone of the phosgenation reactor heated to 340° C. in 4.5 s (that is, the residence time was 4.5 s). Then the mixed gas after reaction was fed to a quenching zone of the phosgenation reactor. Temperature of the mixed gas after reaction was reduced rapidly by utilizing the latent heat of vaporization of the quenching medium (dichlorobenzene), where the flow rate of the quenching medium 1,2-dichlorobenzene (−10° C., 0.6 MPa) was 800 g/h. The target product was cooled to 140° C. and then collected at the bottom of a reaction absorption vessel (collecting vessel). In this process, dichlorobenzene was used to capture and absorb the target product. By weighing and gas chromatography, the conversion rate of the reaction was 100% and the selectivity was 94.5%.

### Example 2-2: HDA was Used as a Raw Material and Liquid HDI at −10° C. was Used as a Quenching Medium

Phosgene and an inert carrier gas were preheated to 340° C. Phosgene at 990 g/h (10 eq) and the inert carrier gas (nitrogen) at 4.48 L/h (that is, 20% by mole of HDA) were mixed and first introduced into a phosgenation reactor. After 10 min, HDA preheated to 340° C. was introduced into the phosgenation reactor at a rate of 116 g/h. The two reactant streams were mixed at the inlet of the reactor. The stream was then flowed through a reaction zone of the phosgenation reactor heated to 340° C. in 4.5 s (that is, the residence time was 4.5 s). Then the mixed gas after reaction was fed to a quenching zone of the phosgenation reactor. Temperature of the mixed gas after reaction was reduced rapidly by utilizing the latent heat of vaporization of the quenching medium (liquid HDI cooled to −10° C.), where the flow rate of the quenching medium liquid HDI (−10° C., 0.5 MPa) was 1050 g/h. The target product was cooled to 140° C. and then collected at the bottom of a reaction absorption vessel (collecting vessel). In this process, liquid HDI cooled to −10° C. was used to capture and absorb the target product. By weighing and gas chromatography, the conversion rate of the reaction was 100% and the selectivity was 93.1%.

### Example 2-3: HDA was Used as a Raw Material, and Liquid Phosgene was Used as a Quenching Medium

Phosgene and an inert carrier gas were preheated to 340° C. Phosgene at 990 g/h (10 eq) and the inert carrier gas (nitrogen) at 4.48 L/h (that is, 20% by mole of HDA) were mixed and first introduced into a phosgenation reactor. After 10 min, HDA preheated to 340° C. was introduced into the phosgenation reactor at a rate of 116 g/h. The two reactant streams were mixed at the inlet of the reactor. The stream was then flowed through a reaction zone of the phosgenation reactor heated to 340° C. in 4.5 s (that is, the residence time was 4.5 s). Then the mixed gas after reaction was fed to a quenching zone of the phosgenation reactor. Temperature of the mixed gas after reaction was reduced rapidly by utilizing the latent heat of vaporization of the quenching medium (liquid phosgene), where the flow rate of the quenching medium liquid phosgene (−10° C., 0.5 MPa) was 800 g/h. The target product was cooled to 140° C. and then collected at the bottom of a reaction absorption vessel (collecting vessel). In this process, liquid phosgene was used to capture and absorb the target product. By weighing and gas chromatography, the conversion rate of the reaction was 100% and the selectivity was 97.2%.

### Example 2-4: HDA was Used as a Raw Material, and Liquid Nitrogen was Used as a Quenching Medium

Phosgene and an inert carrier gas were preheated to 340° C. Phosgene at 990 g/h (10 eq) and the inert carrier gas (nitrogen) at 4.48 L/h (that is, 20% by mole of HDA) were mixed and first introduced into a phosgenation reactor. After 10 min, HDA preheated to 340° C. was introduced into the phosgenation reactor at a rate of 116 g/h. The two reactant streams were mixed at the inlet of the reactor. The stream was then flowed through a reaction zone of the phosgenation reactor heated to 340° C. in 4.5 s (that is, the residence time was 4.5 s). Then the mixed gas after reaction was fed to a quenching zone of the phosgenation reactor. Temperature of the mixed gas after reaction was reduced rapidly by utilizing the latent heat of vaporization of the quenching medium (liquid nitrogen), where the flow rate of the quenching medium liquid nitrogen (−177° C., 0.5 MPa) was 450 g/h. The target product was cooled to 140° C. and then collected at the bottom of a reaction absorption vessel (collecting vessel). In this process, liquid nitrogen was used to capture and absorb the target product. By weighing and gas chromatography, the conversion rate of the reaction was 100% and the selectivity was 94.3%.

### Example 2-5: HDA was Used as a Raw Material, and Liquid Carbon Dioxide was Used as a Quenching Medium

Phosgene and an inert carrier gas were preheated to 340° C. Phosgene at 990 g/h (10 eq) and the inert carrier gas (nitrogen) at 4.48 L/h (that is, 20% by mole of HDA) were mixed and first introduced into a phosgenation reactor. After 10 min, HDA preheated to 340° C. was introduced into the phosgenation reactor at a rate of 116 g/h. The two reactant streams were mixed at the inlet of the reactor. The stream was then flowed through a reaction zone of the phosgenation reactor heated to 340° C. in 4.5 s (that is, the residence time was 4.5 s). Then the mixed gas after reaction was fed to a quenching zone of the phosgenation reactor. Temperature of the mixed gas after reaction was reduced rapidly by utilizing the latent heat of vaporization of the quenching medium (liquid carbon dioxide), where the flow rate of the quenching medium liquid carbon dioxide (−46° C., 0.7 MPa) was 600 g/h. The target product was cooled to 140° C. and then collected at the bottom of a reaction absorption vessel (collecting vessel). In this process, liquid carbon dioxide was used to capture and absorb the target product. By weighing and gas chromatography, the conversion rate of the reaction was 100% and the selectivity was 91.1%.

### Example 2-6: HDA was Used as a Raw Material, and Liquid Hydrogen Chloride was Used as a Quenching Medium

Phosgene and an inert carrier gas were preheated to 340° C. Phosgene at 990 g/h (10 eq) and the inert carrier gas (nitrogen) at 4.48 L/h (that is, 20% by mole of HDA) were mixed and first introduced into a phosgenation reactor. After 10 min, HDA preheated to 340° C. was introduced into the phosgenation reactor at a rate of 116 g/h. The two reactant streams were mixed at the inlet of the reactor. The stream was then flowed through a reaction zone of the phosgenation reactor heated to 340° C. in 4.5 s (that is, the residence time was 4.5 s). Then the mixed gas after reaction was fed to a quenching zone of the phosgenation reactor. Temperature of the mixed gas after reaction was reduced rapidly by utilizing the latent heat of vaporization of the quenching medium liquid hydrogen chloride, where the flow rate of the quenching medium liquid hydrogen chloride (−40° C., 0.7 MPa) was 520 g/h. The target product was cooled to 140° C. and then collected at the bottom of a reaction absorption vessel (collecting vessel). In this process, liquid hydrogen chloride was used to capture and absorb the target product. By weighing and gas chromatography, the conversion rate of the reaction was 100% and the selectivity was 90.1%.

### Example 2-7: HDA Hydrochloride was Used as a Raw Material, and Liquid Phosgene was Used as a Quenching Medium

Phosgene and an inert carrier gas were preheated to 340° C. Phosgene at 990 g/h (10 eq) and the inert carrier gas (nitrogen) at 4.48 L/h (that is, 20% by mole of HAD hydrochloride) were mixed and first introduced into a phosgenation reactor. After 10 min, gaseous HAD hydrochloride preheated to 340° C. was introduced into the phosgenation reactor at a rate of 189 g/h. The two reactant streams were mixed at the inlet of the reactor. The stream was then flowed through a reaction zone of the phosgenation reactor heated to 340° C. in 4.5 s (that is, the residence time was 4.5 s). Then the mixed gas after reaction was fed to a quenching zone of the phosgenation reactor. Temperature of the mixed gas after reaction was reduced rapidly by utilizing the latent heat of vaporization of the quenching medium liquid phosgene, where the flow rate of the quenching medium liquid phosgene (−10° C., 0.5 MPa) was 800 g/h. The target product was cooled to 140° C. and then collected at the bottom of a reaction absorption vessel (collecting vessel). In this process, liquid phosgene was used to capture and absorb the target product. By weighing and gas chromatography, the conversion rate of the reaction was 100% and the selectivity was 95.8%.

### Example 3: Synthesis of IPDI

### Example 3-1: IPDA was Used as a Raw Material, and Liquid Phosgene was Used as a Quenching Medium

Phosgene and an inert carrier gas were preheated to 330° C. Phosgene at 990 g/h (10 eq) and the inert carrier gas (nitrogen) at 4.48 L/h (that is, 20% by mole of IPDA) were mixed and first introduced into a phosgenation reactor. After 10 min, IPDA preheated to 330° C. was introduced into the phosgenation reactor at a rate of 170 g/h. The two reactant streams were mixed at the inlet of the reactor. The stream was then flowed through a reaction zone of the phosgenation reactor heated to 330° C. in 5 s (that is, the residence time was 5 s). Then the mixed gas after reaction was fed to a quenching zone of the phosgenation reactor. Temperature of the mixed gas after reaction was reduced rapidly by utilizing the latent heat of vaporization of the quenching medium liquid phosgene, where the flow rate of the quenching medium liquid phosgene (−10° C., 0.5 MPa) was 850 g/h. The target product was cooled to 130° C. and then collected at the bottom of a reaction absorption vessel (collecting vessel). In this process, liquid phosgene was used to capture and absorb the target product. By weighing and gas chromatography, the conversion rate of the reaction was 100% and the selectivity was 95.3%.

### Example 3-2: IPDA Hydrochloride was Used as a Raw Material, and Liquid Phosgene was Used as a Quenching Medium

Phosgene and an inert carrier gas were preheated to 330° C. Phosgene at 990 g/h (10 eq) and the inert carrier gas (nitrogen) at 4.48 L/h (that is, 20% by mole of IPDA hydrochloride) were mixed and first introduced into a phosgenation reactor. After 10 min, gaseous IPDA hydrochloride preheated to 330° C. was introduced into the phosgenation reactor at a rate of 243 g/h. The two reactant streams were mixed at the inlet of the reactor. The stream was then flowed through a reaction zone of the phosgenation reactor heated to 330° C. in 5 s (that is, the residence time was 5 s). Then the mixed gas after reaction was fed to a quenching zone of the phosgenation reactor. Temperature of the mixed gas after reaction was reduced rapidly by utilizing the latent heat of vaporization of the quenching medium liquid phosgene, where the flow rate of the quenching medium liquid phosgene (−10° C., 0.5 MPa) was 900 g/h. The target product was cooled to 130° C. and then collected at the bottom of a reaction absorption vessel (collecting vessel). In this process, liquid phosgene was used to capture and absorb the target product. By weighing and gas chromatography, the conversion rate of the reaction was 100% and the selectivity was 94.8%.

### Example 4 Synthesis of HTDI

### Example 4-1: HTDA was Used as a Raw Material, and Liquid Phosgene was Used as a Quenching Medium

Phosgene and an inert carrier gas were preheated to 350° C. Phosgene at 990 g/h (10 eq) and the inert carrier gas (nitrogen) at 4.48 L/h (that is, 20% by mole of HTDA) were mixed and first introduced into a phosgenation reactor. After 10 min, HTDA preheated to 350° C. was introduced into the phosgenation reactor at a rate of 128 g/h. The two reactant streams were mixed at the inlet of the reactor. The stream was then flowed through a reaction zone of the phosgenation reactor heated to 350° C. in 5.5 s (that is, the residence time was 5.5 s). Then the mixed gas after reaction was fed to a quenching zone of the phosgenation reactor. Temperature of the mixed gas after reaction was reduced rapidly by utilizing the latent heat of vaporization of the quenching medium liquid phosgene, where the flow rate of the quenching medium liquid phosgene (−10° C., 0.5 MPa) was 850 g/h. The target product was cooled to 150° C. and then collected at the bottom of a reaction absorption vessel (collecting vessel). In this process, liquid phosgene was used to capture and absorb the target product. By weighing and gas chromatography, the conversion rate of the reaction was 100% and the selectivity was 96.3%.

### Example 4-2: HTDA Hydrochloride was Used as a Raw Material, and Liquid Phosgene was Used as a Quenching Medium

Phosgene and an inert carrier gas were preheated to 350° C. Phosgene at 990 g/h (10 eq) and the inert carrier gas (nitrogen) at 4.48 L/h (that is, 20% by mole of HTDA hydrochloride) were mixed and first introduced into a phosgenation reactor. After 10 min, gaseous HTDA hydrochloride preheated to 350° C. was introduced into the phosgenation reactor at a rate of 164 g/h. The two reactant streams were mixed at the inlet of the reactor. The stream was then flowed through a reaction zone of the phosgenation reactor heated to 350° C. in 5.5 s (that is, the residence time was 5.5 s). Then the mixed gas after reaction was fed to a quenching zone of the phosgenation reactor. Temperature of the mixed gas after reaction was reduced rapidly by utilizing the latent heat of vaporization of the quenching medium liquid phosgene, where the flow rate of the quenching medium liquid phosgene (−10° C., 0.5 MPa) was 900 g/h. The target product was cooled to 150° C. and then collected at the bottom of a reaction absorption vessel (collecting vessel). In this process, liquid phosgene was used to capture and absorb the target product. By weighing and gas chromatography, the conversion rate of the reaction was 100% and the selectivity was 96.0%.

Some embodiments of the present invention have been described above. However, it should be clearly pointed out that the present invention is not limited to those embodiments, and additions and modifications made to the disclosure explicitly described in the present invention are intended to be embraced in the scope of the present invention. Moreover, it should be understood that without departing from the spirit and scope of the present invention, the features of various embodiments described in the present invention are not mutually exclusive, and may exist in various combinations and arrangements, even if such combinations and arrangements are not explicitly expressed. Given that certain embodiments of the method for preparing an isocyanate have been described, it will become apparent to those skilled in the art that other embodiments including the concepts of the present application can be used. Therefore, the present application is not limited to certain embodiments, but defined by the spirit and scope of the appended claims.

What is claimed is:

1. A method for preparing an isocyanate, comprising the following steps:
(a) providing a reactant amine stream and a phosgene stream, being passed through and reacting in a reaction zone of a first vessel at a temperature of 200° C.˜600° C., to obtain a reaction product mixture comprising isocyanate, hydrogen chloride and unreacted phosgene;
(b) contacting the reaction product mixture obtained in Step (a) with a first quenching medium stream introduced into a quenching zone of the first vessel, and introducing, via an inlet of a second vessel connected to the quenching zone of the first vessel, the reaction product mixture into the second vessel;
(c) the second vessel of Step (b) comprises a collecting zone and a washing zone, collecting isocyanate in the collecting zone, and passing the hydrogen chloride, unreacted phosgene, and uncollected isocyanate through the washing zone; and
(d) introducing a second quenching medium stream into the washing zone of the second vessel of Step (c), and contacting the hydrogen chloride, unreacted phosgene and uncollected isocyanate of Step (c) with the second quenching medium stream in the washing zone,
wherein the first quenching medium and the second quenching medium are independently selected from the group consisting of isocyanate, phosgene, hydrogen chloride, an inert carrier gas, and any combination thereof, and the ratio of flow rates of the first quenching medium to the phosgene stream of Step (a) is from 0.4:1 to 2:1.

2. A method for preparing isocyanate, comprising the following steps:
(a) providing a reactant amine stream and a phosgene stream, being passed through and reacting in a reaction zone of a first vessel at a temperature of 200° C.˜600° C., to obtain a reaction product mixture comprising isocyanate, hydrogen chloride and unreacted phosgene;
(b) contacting the reaction product mixture obtained in Step (a) with a first quenching medium stream introduced into a quenching zone of the first vessel, and introducing, via an inlet of a second vessel connected to the quenching zone of the first vessel, the reaction product mixture into the second vessel;
(c) the second vessel of Step (b) comprises a collecting zone and a washing zone, collecting isocyanate in the collecting zone, and passing the hydrogen chloride, unreacted phosgene, and uncollected isocyanate through the washing zone; and
(d) introducing a second quenching medium stream into the washing zone of the second vessel of Step (c), and contacting the hydrogen chloride, unreacted phosgene and uncollected isocyanate of Step (c) with the second quenching medium stream in the washing zone,
wherein the first quenching medium is phosgene, and the second quenching medium is selected from the group consisting of isocyanate, phosgene, hydrogen chloride, an inert carrier gas, and any combination thereof.

3. The method according to claim 1 or 2, wherein the reactant amine stream of Step (a) is preheated to 200° C.˜600° C. by a first pre-heater before being passed into the first vessel; and/or the phosgene stream of Step (a) is preheated to 200° C.˜600° C. by a second pre-heater before being passed into the first vessel.

4. The method according to any one of the preceding claims, wherein the reactant amine stream and/or the phosgene stream of Step (a) exist(s) in a gaseous or atomized form before, during or after being passed into the first vessel.

5. The method according to any one of the preceding claims, wherein the reactant amine stream and the phosgene stream of Step (a) are mixed in a headspace of the first vessel.

6. The method according to any one of the preceding claims, wherein the reactant amine stream and the phosgene stream of Step (a) flow top-down in the reaction zone of the first vessel, and react during this process, to obtain the reaction product mixture comprising isocyanate, hydrogen chloride and unreacted phosgene.

7. The method according to any one of the preceding claims, wherein the reactant amine stream and the phosgene stream of Step (a) have a retention time of not more than 260 seconds in the reaction zone of the first vessel.

8. The method according to any one of the preceding claims, wherein the phosgene stream of Step (a) is stoichiometric excess based on amino groups of the reactant amine stream.

9. The method according to any one of the preceding claims, wherein feed ratio (by mole) of the phosgene stream and the reactant amine stream of Step (a) is from 7:1 to 15:1.

10. The method according to any one of the preceding claims, wherein the phosgene stream and/or the reactant amine stream of Step (a) is/are passed into the first vessel simultaneously or sequentially with an inert carrier gas.

11. The method according to claim 10, wherein the inert carrier gas is preheated to 200° C.˜600° C. before being passed into the first vessel.

12. The method according to claim 10 or 11, wherein molar flow rate of the inert carrier gas is 10-100% of molar flow rate of the reactant amine stream or the phosgene stream.

13. The method according to any one of the preceding claims, wherein the first quenching medium of Step (b) and the second quenching medium of Step (d) are the same.

14. The method according to any one of claims 1 to 12, wherein the first quenching medium of Step (b) and the second quenching medium of Step (d) are different.

15. The method according to any one of the preceding claims, wherein the first quenching medium of Step (b) and/or the second quenching medium of Step (d) comprise(s) no organic solvent.

16. The method according to any one of the preceding claims, wherein the first quenching medium of Step (b) is liquid phosgene.

17. The method according to any one of the preceding claims, wherein the second quenching medium is a liquid.

18. The method according to claim 17, wherein both the first quenching medium and the second quenching medium are liquid phosgene.

19. The method according to any one of the preceding claims, wherein in Step (b), temperature of the reaction product mixture obtained in Step (a) is reduced rapidly by utilizing latent heat of vaporization of the first quenching medium.

20. The method according to any one of the preceding claims, wherein in Step (d), the flowing directions of the hydrogen chloride, unreacted phosgene and uncollected isocyanate of Step (c) and the second quenching medium stream are opposite in the washing zone.

21. The method according to claim 20, wherein the washing conditions are configurated to allow the hydrogen chloride and unreacted phosgene (and optionally, inert carrier gas) overflowing from top of the second vessel, and the uncollected isocyanate flowing back to the collecting zone of the second vessel.

22. The method according to claim 21, wherein the hydrogen chloride and unreacted phosgene (and optionally, inert carrier gas) overflowing from top of the second vessel are cooled to −5˜20° C. by a cooler, and then passed through a pressure control system.

23. The method according to claim 21 or 22, wherein the washing condition is controlling temperature of the second quenching medium and/or the cooler within a range of 0˜15° C.

24. The method according to any one of claims 21 to 23, wherein the hydrogen chloride overflowing from top of the second vessel is subjected to hydrogen chloride refining after being passed through the pressure control system, to form by-product hydrochloric acid.

25. The method according to any one of claims 21 to 23, wherein the phosgene overflowing from top of the second vessel is recycled, to form the phosgene stream of Step (a) or the first quenching medium stream of Step (b).

26. The method according to any one of the preceding claims, wherein the isocyanate is a diisocyanate.

27. The method according to any one of the preceding claims, wherein the isocyanate is an aliphatic diisocyanate or an aromatic diisocyanate.

28. The method according to any one of the preceding claims, wherein the isocyanate is selected from the group consisting of methylene diphenyl diisocyanate as a pure isomer or as a mixture of isomers, toluene diisocyanate as a pure isomer or as a mixture of isomers, 2,6-xylyl isocyanate, 1,5-naphthalene diisocyanate, methyl isocyanate, ethyl isocyanate, propyl isocyanate, isopropyl isocyanate, butyl isocyanate, isobutyl isocyanate, t-butyl isocyanate, pentyl isocyanate (e.g., pentamethylene diisocyanate), t-pentyl isocyanate, isopentyl isocyanate, neopentyl isocyanate, hexyl isocyanate (e.g., hexamethylene diisocyanate), cyclopentyl isocyanate, cyclohexyl isocyanate, and phenyl isocyanate (e.g., p-phenylene diisocyanate).

29. The method according to any one of the preceding claims, wherein the isocyanate is PDI, HDI, IPDI or HTDI.

30. The method according to any one of the preceding claims, wherein the reactant amine has a structural formula of R(NH₂)ₙ, wherein n is 1, 2 or 3, and R is an aliphatic or aromatic hydrocarbyl group.

31. The method according to claim 30, wherein n is 2, and R is an aliphatic hydrocarbyl group.

32. The method according to claim 31, wherein n is 2, and R is an aliphatic hydrocarbyl group having 2-10 carbon atoms.

33. The method according to claim 32, wherein n is 2, and R is a linear or cyclic aliphatic hydrocarbyl group having 3-10 carbon atoms.

34. The method according to any one of the preceding claims, wherein the reactant amine exists in a free form.

35. The method according to any one of the preceding claims, wherein the reactant amine exists as an amine salt.

36. The method according to claim 35, wherein the amine salt is selected from the group consisting of a hydrochloride, a sulfate, a bisulfate, a nitrate, and a carbonate.

37. The method according to any one of the preceding claims, wherein the reactant amine is one or more selected from the group consisting of ethyl amine, butyl amine, pentamethylene diamine, hexamethylene diamine, 1,4-diamino butane, 1,8-diamino octane, aniline, p-phenylene diamine, m-xylylene diamine, toluene diamine, 1,5-naphthalene diamine, diphenylmethane diamine, dicyclohexylmethane diamine, m-cyclohexyldimethylene diamine, isophorone diamine, methyl cyclohexane diamine, and trans-1,4-cyclohexane diamine.

38. The method according to any one of the preceding claims, wherein the reactant amine is selected from the group consisting of PDA, PDA hydrochloride, HDA, HDA hydrochloride, IPDA, IPDA hydrochloride, HTDA and HTDA hydrochloride, the first quenching medium is liquid phosgene, and the second quenching medium is selected from the group consisting of liquid PDI, liquid HDI, liquid phosgene, liquid nitrogen, liquid carbon dioxide and liquid hydrogen chloride.

39. The method according to claim 38, wherein both the first quenching medium and the second quenching medium are liquid phosgene.

40. The method according to any one of claims 2 to 39, wherein the ratio of flow rates of the phosgene as the first quenching medium to the phosgene stream of Step (a) is from 0.7:1 to 1.2:1.
